(19) United States
(12) Patent Application Publication
(10) Pub. No.: US 2026/0151179 A1
(43) Pub. Date: Jun. 4, 2026

(54) DEVICES, SYSTEMS AND METHODS OF MEASURING IMPEDANCE OF AN ELECTRODE

(71) Applicant: St. Jude Medical, Cardiology Division, Inc., St. Paul, MN (US)

(72) Inventors: Hong Cao, Maple Grove, MN (US); Jeffrey A. Schweitzer, St. Paul, MN (US); Peter C. Mooers, Brooklyn Park, MN (US); Scott C. Meyerson, Ham Lake, MN (US)

(21) Appl. No.: 19/406,233

(22) Filed: Dec. 2, 2025

Related U.S. Application Data
(60) Provisional application No. 63/727,366, filed on Dec. 3, 2024.

(51) Int. Cl. A61B 18/14; A61B 5/00; A61B 18/00; A61B 34/20

(52) U.S. Cl. CPC A61B 18/14; A61B 5/7225; A61B 34/20; A61B 2018/00827; A61B 2018/00892; A61B 2034/2053

(57) ABSTRACT

A medical device system includes a reference electrode, a drive circuitry configured to provide drive signal, a medical device having at least one electrode located on the medical device, and sense circuitry having a first channel and a second channel. The at least one electrode is connected to receive the drive signal from the drive circuitry. The first channel compares a first voltage measured at the at least one electrode and a reference voltage measured at the reference electrode and the second channel provides a return path for the drive circuitry.

### CROSS-REFERENCE TO RELATED APPLICATIONS

This application claims the benefit of and priority to U.S. provisional application 63/727,366, titled “DEVICES, SYSTEMS AND METHODS OF MEASURING IMPEDANCE OF AN ELECTRODE”, filed Dec. 3, 2024, the contents of which are incorporated by reference herein.

### TECHNICAL FIELD

The subject matter disclosed herein relates to medical devices and, in particular, to systems and methods of measuring an impedance associated with an electrode.

### BACKGROUND

Measuring the impedance between pairs of electrodes located on a medical device may be utilized to detect contact between the pairs of electrodes and tissue. A drive signal is applied to the pair of electrodes and sensing circuitry is utilized to monitor the respective impedance. In some embodiments, the sensing circuitry utilizes a pair of channels to measure the respective impedance. The first channel is connected to compare the voltage associated with a first electrode with a reference voltage provided by a surface patch electrode and the second channel is connected to compare the voltage associated with the second electrode with the reference voltage provided by the surface patch electrode. The difference in voltages measured by the first and second channel may be utilized to calculate the impedance between the first and second electrodes, with the impedance being utilized to make determinations regarding contact status. The sense circuitry utilized to measure the impedance between the respective electrodes can be described as a three-terminal device based on the voltage measured at each electrode and the reference voltage provided by the surface patch electrode. In some applications the impedance measured by a three-terminal device does not provide the desired accuracy. For these applications, it would be beneficial to develop an impedance measurement system that provides improved accuracy.

### SUMMARY

According to some aspects, the claimed invention provides a system and method of making two-terminal impedance measurements using a first channel connected to a first electrode and a reference voltage associated with a reference electrode. The second channel is kept “open” (i.e., NOT connected to an electrode). However, the second channel provides a return path for the drive circuitry despite not being connected to a second electrode. In some embodiments, the second channel is connected to the first channel through a shunt resistor. In other embodiments, no shunt resistor is required between the first and second channel. In some embodiments the two-terminal impedance measurements can utilize the same circuitry as that utilized in the three-terminal impedance measurements, with one of the only changes being whether the second channel is connected to a second electrode (i.e., three-terminal configuration) or kept “open” (i.e., two-terminal configuration). A typical three-terminal impedance measurement uses a first channel connected to a first electrode but connects the second channel to the second electrode, with the connection of each channel to a reference electrode making this a three-terminal device.

In some embodiments, the two-terminal impedance measurement is utilized for sheath detection (i.e., to determine whether the first electrode associated with a medical device is located within a sheath utilized to deliver the medical device or external to the sheath. In general, the two-impedance measurement is larger when the first electrode is located within the sheath and smaller when the first electrode is located external to the sheath. In some embodiments, the two-terminal impedance measurement may be utilized for additional detecting tissue contact/proximity.

According to one aspect, a medical device system includes a reference electrode, drive circuitry configured to provide drive signal, a medical device having a first electrode located on the medical device, the first electrode being connected to receive the drive signal from the drive circuitry, and sense circuitry having a first channel and a second channel, the first channel comparing a first voltage measured at the first electrode and a reference voltage measured at the reference electrode and the second channel providing a return path for the drive circuitry.

The drive signal supplied to the first electrode may return to the drive circuitry through the reference electrode and the second channel.

The second channel may be connected to the first channel via a shunt resistor.

The first channel may include an amplifier having a first input connected to monitor voltage on the first electrode and a second input connected to the reference voltage.

The output of the amplifier may be representative of impedance between the first electrode and the reference electrode.

The second channel may include a second amplifier having a first input connected to the reference electrode through a ground isolation terminal and a second input connected to the reference voltage.

A circuit path may exists from the drive circuitry, to the first electrode, to the reference electrode, to a return on the drive circuitry via a designed return path between the first channel and the second channel.

The first electrode may be located on a shaft of the medical device.

The first electrode may be located on a spline of a basket assembly.

The first electrode may be included as part of an array of electrodes.

A second drive circuit may provide a second drive signal, a third channel and a fourth channel, wherein the third channel may be connected to the first electrode and the fourth channel may be connected to a second electrode located on the medical device, wherein the second drive signal may be provided to both the first electrode and the second electrode and wherein the third channel and the fourth channel make a three-terminal impedance measurement.

The reference electrode may be a surface patch electrode.

The reference electrode may be a second electrode located on the medical device.

A two-terminal impedance measurement circuit includes drive circuitry configured to provide a drive signal and sense circuitry having a first channel and a second channel, the first channel measuring impedance between a first electrode located on a medical device and a reference electrode and the second channel providing a designed return path for the drive circuitry without the second channel being connected to any electrodes on the medical device.

The drive signal may be provided to the first electrode is provided at a unique frequency.

The second channel may be connected to the first channel via a shunt resistor.

The first channel may include an amplifier having a first terminal connected to monitor voltage on the first electrode and a second terminal connected to a reference voltage associated with the reference electrode.

The second channel may provide a return path for the drive circuitry via the reference electrode and ground reference shared by the reference electrode and the second channel.

The reference electrode may be a surface patch electrode.

The reference electrode may be a second electrode located on the medical device.

A method of making a two-terminal measurement using a sense circuitry configured for three-terminal impedance measurements includes providing, using drive circuitry, a first drive signal to a first electrode located on a medical device. The method further includes measuring, using a first channel a voltage between the first electrode and a reference electrode and providing, using a second channel, a return path for the drive circuitry, wherein the second channel is not connected to any electrodes located on the medical device.

The second channel may be connected to the first channel via a shunt resistor.

The reference electrode may be a surface patch electrode.

The reference electrode may be a second electrode located on the medical device.

### BRIEF DESCRIPTION OF THE DRAWINGS

FIG. 1 is a diagrammatic depiction of a system including a medical device for insertion within a patient, the system configured to utilize a two-terminal impedance measurement to determine proximity or contact status of the one or more electrodes located at a distal end of the medical device according to some embodiments.

FIG. 2 is a circuit diagram of a three-terminal impedance measurement circuit connected to first and second electrodes located on the medical device as known in the prior art.

FIG. 3 is a circuit diagram of a two-terminal impedance measurement circuit connected to a first electrode located on the medical device according to some embodiments.

FIG. 4A is a circuit diagram illustrating first and second channels connected to make a two-terminal measurement with respect to a first electrode and third and fourth channels connected to make a two-terminal measurement with respect to a second electrode according to some embodiments.

FIG. 4B is a circuit diagram illustrating first and second channels connected to make a two-terminal measurement with respect to a first electrode and third and fourth channels connected to make a three-terminal measurement with respect to the first electrode and a second electrode according to some embodiments.

FIGS. 5A-5D illustrate a medical device having a plurality of shaft electrodes at various stages of sheathing the medical device within a sheath according to some embodiments.

FIG. 6A is a graph illustrating sheath detection using three-terminal measurements associated with pairs of electrode located on a catheter shaft as known in the prior art.

FIG. 6B is a graph illustrating sheath detection using two-terminal measurements associated with electrodes located on a catheter shaft according to some embodiments.

FIG. 7 is an oblique view of a distal end of a catheter having a plurality of splines in a basket configuration wherein at least one electrode located on one of the splines according to some embodiments.

FIG. 8 is a graph comparing three-terminal measurements with two-terminal measurements associated with spline electrodes located on the plurality of splines according to some embodiments.

FIG. 9 is a top view of a distal end of a catheter having a plurality of splines located in a plane according to some embodiments.

FIG. 10 is a graph comparing three-terminal measurements with two-terminal measurements associated with spline electrodes according to some embodiments.

FIG. 11 is a flowchart illustrating a method of detecting sheath position and/or tissue proximity based on one or more two-terminal measurements according to some embodiments.

### DETAILED DESCRIPTION

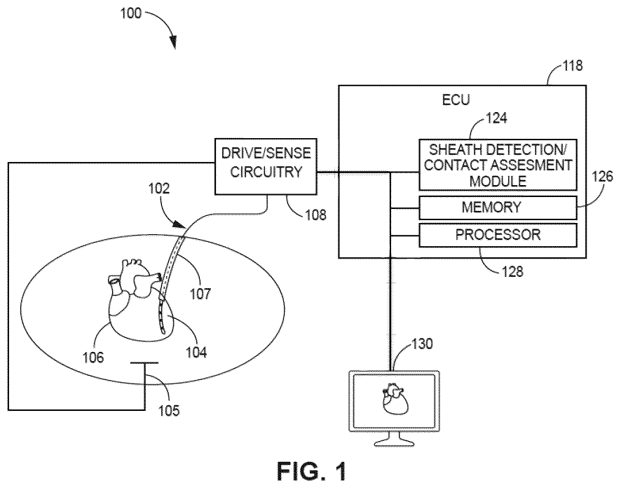

FIG. 1 is a diagrammatic depiction of a system 100 including a medical device 102 for insertion within a patient, the system configured to utilize a two-terminal impedance measurement to determine proximity or contact status of the one or more electrodes located at a distal end of the medical device according to some embodiments. In some embodiments, the system 100 includes drive and sense circuitry 108 and an electronic control unit (ECU) 118 that includes memory 126 and a processor 128, wherein instructions stored by the memory 126 are executed by the processor 128 to implement sheath detection/contact assessment detector 124. The system 100 may also include a display 130 for displaying information to the technician/doctor, which may include sheath detection information and/or tissue proximity/contact status information derived from the two-terminal measurements. In some embodiments, one or more reference electrodes 105 may be utilized. For example, in some embodiments the reference electrode 105 is a surface patch electrode adhered to the skin of the patient.

In some embodiments, the medical device 102 is an elongate medical device, such as a diagnostic and/or therapy catheter, an introducer, sheath, or other similar type of device. The medical device 102 includes a distal end 104 and a proximal end (not shown) that includes a handle operated by an operator as well as interfaces for interfacing the medical device 102 to the drive/sense circuitry 108. The distal end 104 may include various sensors and/or components for localization/navigation of the distal end 104 within the patient, mapping of physiological parameters within the patient, and delivery of therapy. In particular, the distal end 104 of the medical device includes a plurality of electrodes that may be utilized for one or more of these purposes. In some embodiments, the distal end 104 of the medical device is communicated to a location within the patient via a sheath 107, wherein the distal end 104 is extended from within the sheath 107. As described in more detail below, it is desirable to be able to determine whether the distal end 104 has exited the sheath 107 and also the proximity of the electrodes to the adjacent tissue.

In some embodiments, sheath detection and/or contact status of the one or more electrodes located at the distal end 104 of the medical device 102 is determined based on one or more electrical characteristics measured at the electrode. For example, in some embodiments the measured electrical characteristic is a two-terminal impedance measurement generated in response to a drive or excitation signal being provided to a first electrode. The resulting voltage is measured relative to a reference voltage (e.g., from a patch electrode), wherein the measured voltage is correlated with an impedance associated with the first electrode. In some embodiments, sheath detection/contact assessment module 124 utilizes the two-terminal impedance measurement, either alone or in combination with other measured electrical characteristics, to determine the sheath status/contact status of each electrode. In some embodiments, the sheath status refers to a determination of whether a particular electrode associated with distal end 104 is located within the sheath 107 or whether the particular electrode is associated with a portion of the distal end 104 that has been unsheathed (i.e., exited the sheath 107). In some embodiments this is a binary determination. However, in some embodiments the two-terminal impedance measurement corresponds with location of the electrode within the sheath. In some embodiments, the two-terminal impedance measurement may also be utilized to determine contact status of the electrode with respect to adjacent tissue. In some embodiments, the term “contact status” is a binary determination, with the electrode either being “in contact” with the tissue or “not in contact” with the tissue. In other embodiments, the term “contact status” may include additional contact states, such as “intermittent contact”. In still other embodiments, the term “contact status” may describe a proximity of the electrode to adjacent tissue, wherein the two-terminal impedance measurement may be correlated with a distance of the electrode from the adjacent tissue.

In the embodiment shown in FIG. 1, drive/sense circuitry 108 is utilized to generate the drive or excitation signal. In some embodiments, drive/sense circuitry 108 generates one or more excitation or drive signals, each at a unique frequency. More specifically, the drive/sense circuitry 108 may generate a plurality of excitation or drive signals having unique frequencies within a range from about 1 kHz to over 500 kHz, more typically within a range of about 2 kHz to 200 kHz, and even more typically between about 10 kHz and about 20 kHz, in one embodiment. The drive signals may each have a constant current, typically in the range of between 1-200 μA, and more typically about 5 μA, in one embodiment. The drive/sense circuitry 108 may also generate signals involved in, for example, determining a location of the electrodes within the body of the patient that may be utilized for mapping, navigation, and/or therapy delivery. In some embodiments, a portion of the drive/sense circuitry 110 is implemented by ECU 112. In these embodiments, one or more digital-to-analog (D/A) converters may be utilized to convert a digital signal generated by the ECU 112 to an analog signal delivered to the electrodes located at the distal end 108 of the medical device 102. In some embodiments, the drive signal may be applied to a plurality of different electrodes located at the distal end 108 and may require additional circuitry (not shown) such as a switch for selectively delivering the drive signal to the selected electrodes or modulator for generating drive signals at different frequencies.

In response to the drive signals supplied to the selected electrodes, the drive/sense circuitry 110 monitors a resulting voltage generated at one of the plurality of electrodes. In some embodiments, the voltage at a selected electrode is measured with respect to a reference electrode 105, such as a reference electrode associated with a surface patch or reference electrode located on the medical device 102 or separate medical device (not shown) located in the patient's body. In some embodiments, sensed voltages are converted to a digital signal by an analog-to-digital (A/D) converter (not shown) and provided to the ECU 118 for further processing. Once again, in some embodiments additional components such as a switch and/or synchronous demodulator associated with the drive/sense circuitry 110 or ECU 118 may be utilized to select the sense electrodes or signals to be analyzed.

In some embodiments, the memory 126 may be configured to store data respective of the medical device 102, the patient, and/or other data (e.g., calibration data). Such data may be known before a medical procedure (medical device specific data, number of catheter electrodes, etc.), or may be determined and stored during a procedure. The memory 126 may also be configured to store instructions that, when executed by the processor 128 and/or a sheath detection/contact assessment module 124, cause the ECU 118 to perform one or more methods, steps, functions, or algorithms described herein. For example, but without limitation, the memory 126 may include data and instructions for determining impedances respective of one or more electrodes on the medical device 102 and utilizing the impedance measurements to determine a sheath position and/or contact status of the one or more electrodes. In some embodiments, the sheath detection/contact assessment module 124 utilizes a processor executing instructions stored on the memory 126, an application specific integrated circuit (ASIC), or other type of processor. The ECU may be connected to a display 130, which may display an output of sensed tissue (e.g., heart), the medical device (not shown) and/or determined contact status of the one or more electrodes of the medical device 102.

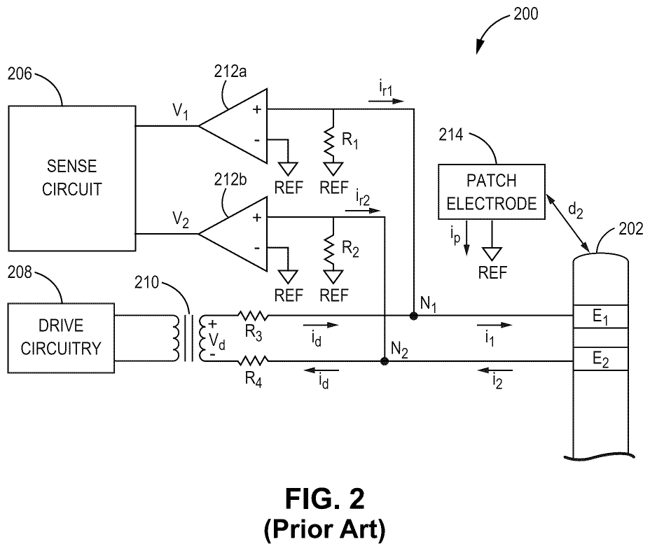

FIG. 2 is a circuit diagram of a three-terminal impedance measurement circuit 200 connected to first and second electrodes E₁, E₂ located on the medical device 202 as known in the prior art. The three-terminal impedance measurement circuit 200 includes sense circuitry 206, drive circuitry 208, isolation/transformer 210, first and second operational-amplifier (hereinafter amplifiers) 212a, 212b, and a reference electrode 214 (e.g., surface patch electrode). The drive circuitry 208 provides a drive current to electrodes E₁, E₂ and sense circuitry 206 measures the voltage V₁, V₂ at each electrode E₁, E₂, respectively. The measured voltages can be utilized to determine an impedance between the respective electrodes E₁ and E₂ that in turn is utilized to determine, for example, contact status between the electrodes E1, E2 and adjacent tissue and/or sheath detection.

Drive circuitry 208 is connected to isolation/transformer 210 to generate a drive current i_{d}. Sense circuitry 206 is connected to monitor the voltage at nodes N₁, N₂ by comparing the voltage monitored at each node to a reference voltage V_{ref}. The reference voltage may be provided by a reference electrode located on the medical device 202 or a reference electrode affixed to the skin of the patient (i.e., surface patch electrode). First amplifier 212a receives at the non-inverting input the voltage monitored at node N₁, which corresponds with the voltage at first electrode E₁, and receives at the inverting input the reference voltage V_{ref}. The output voltage V₁ provided at the output of the first amplifier 212a is reflective of the difference in voltage between the two inputs. The circuitry utilized to monitor the voltage at the first node N₁ is referred to as a first channel. Second amplifier 212b receives at the non-inverting input the voltage monitored at node N₂, which corresponds with the voltage at second electrode E₂, and receives at the inverting input the reference voltage V_{ref.} The output voltage V₂ provided at the output of the second amplifier 212b is reflective of the difference in voltage between the two inputs. The circuitry utilized to monitor the voltage at the second node N₂ is referred to as a second channel. The voltages V₁ and V₂ —because they are both measured with respect to the reference voltage V_{ref} defined by the patch electrode 214—reflect the voltage difference between electrodes E₁ and E₂, respectively, and can be utilized to calculate the impedance between the respective electrodes E₁, E₂. Because the sense circuit relies on voltages measured at nodes N₁, N₂, and the reference voltage V_{ref}, the measurement is referred to as a three-terminal measurement.

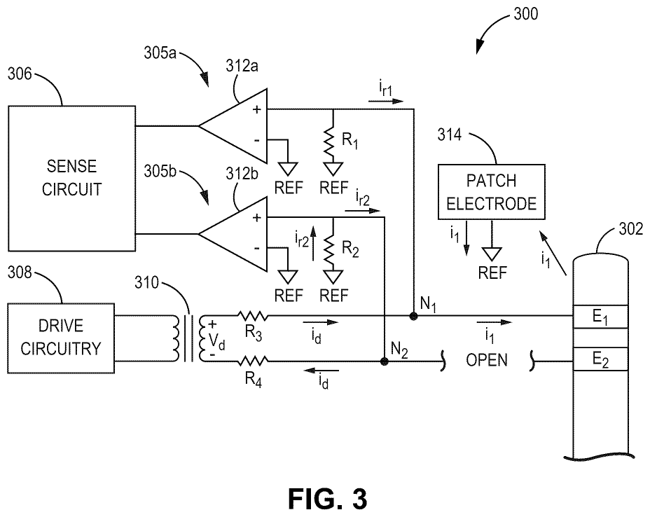

The currents through the respective branches of the circuits are labeled in the embodiment shown in FIG. 2 to illustrate the differences between the three-terminal measurement shown in FIG. 2 and the two-terminal measurement shown in FIG. 3. The drive current i_{d} provided by isolation/transformer 210 is equal on both the top of the transformer 210 (through resistor R3) and on the bottom of the transformer 210 (through resistor R4). First electrode E₁ is connected to the top of the transformer and receives a drive current labeled i₁ is provided to electrode E₁. The second electrode E₂ is connected to the bottom of the transformer and provides return current i₂. Although most of the current provided to first electrode E₁ and returns via the second electrode E₂, at least a portion of the current provided to the first and second electrodes E₁ and E₂ leak to patch electrode 214, resulting in patch current iₚ and through the reference node V_{ref}.

FIG. 3 is a circuit diagram of a two-terminal impedance measurement circuit 300 connected to a first electrode E₁ located on the medical device 302 according to some embodiments. The two-terminal impedance measurement circuit 300 includes sense circuit 306, drive circuitry 308, isolation/transformer 310, first and second amplifiers 312a, 312b, and a reference electrode 314. The two-terminal impedance measurement circuit 300 once again includes two channels; a first channel 305a is connected at first node N₁ to monitor the voltage at the first electrode E₁ and a second channel 305b is connected at second node N₂. As discussed in more detail below the second node N₂ is not connected to any other electrodes on the medical device 302. The reference electrode 314 may be a patch electrode located on the surface of the patient or may be a reference electrode located on medical device 302.

The drive circuitry 308 provides a drive current i_{d} that flows from the top of the transformer 310 through resistor R3 and returns to the bottom of the transformer 310 via resistor R4. In contrast with the three-terminal measurement circuit, the bottom of the transformer 310 is not connected to a second electrode. The presence of a second electrode shown in FIG. 3 illustrates the open-circuit condition between the drive circuitry 308 and the second electrode, but the second electrode is not required and is not utilized by the two-terminal measurement circuit 300. In the embodiment shown in FIG. 3, there is an open circuit between the second node N₂ and the second electrode E₂. A return path for the current is provided in the two-terminal impedance measurement circuit 300 nonetheless via the second channel 305b. In particular, the drive current i₁ provided to first electrode E₁ does not flow to the second electrode E₂ because of the open circuit condition. Instead, the drive current i₁ provided to the first electrode E₁ is provided a designed return path through the patch electrode 314 and the second channel 305b as indicated by the flow of current i₁ shown in FIG. 3. Current flows through the first electrode E₁ to the reference electrode 314 and through the reference voltage node V_{ref} through resistor R2 of the second channel 305b and back to the second node N₂. As a result, the magnitude of current iᵣ₂ through the second resistor R2 is equal to the drive current i_{d}.

In some embodiment, the first channel 305a monitors the voltage at the first node N₁ with reference to the reference voltage V_{ref} defined by the reference electrode 314. For example, in the embodiment shown in FIG. 3, the non-inverting input of the amplifier 312a is connected to the first node N₁ and the inverting input of the amplifier 312a is connected to the reference voltage V_{ref}. The output voltage V₁ of the amplifier 312a is representative of the voltage between the first electrode and the reference electrode and can be utilized to calculate an impedance between the first electrode E₁ and the patch electrode 314. Because the output is based only on the voltage monitored at the first electrode and the reference voltage associated with the reference electrode 314, this measurement is referred to as a two-terminal impedance measurement. As described in more detail below, the two-terminal impedance measurement may be utilized to detect the position of the medical device 302 within a sheath (e.g., sheath 107 as shown in FIG. 1) and more specifically may be utilized to detect the position of the first electrode E₁ within the sheath. In some embodiments, the two-terminal impedance measurement may be utilized to determine the position of the first electrode E₁ with respect to adjacent tissue (i.e., tissue proximity and/or contact).

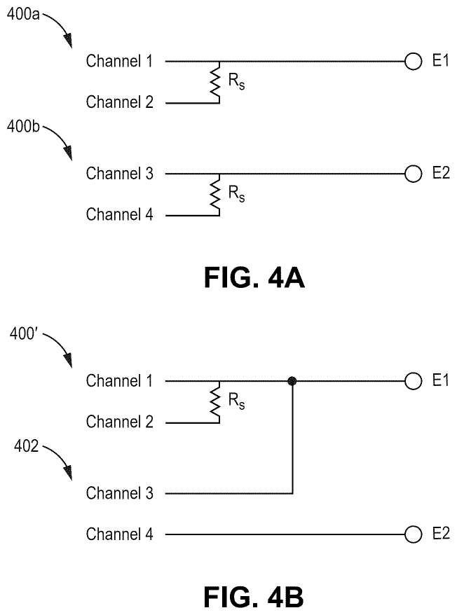

In some embodiments, a shunt resistor (not shown in FIG. 3, but shown in FIGS. 4A and 4B) is connected between the first channel 305a and the second channel 305b. For example, the shunt resistor may be connected between nodes N₁ and N₂. In some embodiments, the shunt resistor is a very large resistance (e.g., 5 kΩ-500 kΩ). One of the benefits of utilizing the shunt resistor is that it provides a more controlled resistive path so that two-terminal measurements are more consistent among a plurality of channels.

In some embodiments, a switch may be located between the second N₂ and a second electrode E₂ to allow the open circuit condition to be selectively initiated by opening the switch. Closing the switch would connect the drive circuitry 308 to the second electrode and provide a return path similar to that shown in FIG. 2 for a typical three-terminal measurement.

FIG. 4A is a circuit diagram illustrating first and second two-terminal measurement circuits 400a, 400b connected to make two-terminal impedance measurements with respect to a first and second electrodes E₁ and E₂, respectively. The first and second two-terminal measurement circuits 400a, 400b may be implemented utilizing the two-terminal measurement circuit 300 described with respect to FIG. 3. In the embodiment shown in FIG. 4A, a first two-terminal measurement circuit 400a utilizes first and second channels to measure a two-terminal impedance associated with electrode E₁ and a second two-terminal measurement circuit 400b utilizes third and fourth channels to measure a two-terminal impedance associated with electrode E₂. In some embodiments, a shunt resistor Rₛ is located between the respective channels of both the first and second two-terminal measurement circuit 400a, 400b, respectively. In particular, FIG. 4A illustrates the requirement of both a first and second channel to make a single two-terminal impedance measurement with respect to a given electrode. Likewise, making a second two-terminal impedance measurement with respect to a second electrode requires two additional channels.

FIG. 4B is a circuit diagram illustrating a two-terminal measurement circuit 400′ connected to make a two-terminal measurement with respect to a first electrode E₁ and a three-terminal measurement circuit 402 connected to make a three-terminal measurement with respect to first and second electrodes E₁ and E₂. The two-terminal measurement circuit 400′ may be implemented utilizing the two-terminal measurement circuit 300 described with respect to FIG. 3. The two-terminal impedance measurement circuit 400′ utilizes first and second channels to measure a two-terminal impedance associated with electrode E₁. The three-terminal measurement circuit 402 may be implemented with the three-terminal measurement circuit 200 described with respect to FIG. 2. The three-terminal measurement circuit 402 utilizes third and fourth channels to measure a three-terminal impedance associated with electrodes E₁ and E₂. In the embodiment shown in FIG. 4B, electrode E₁ is utilized both as part of a two-terminal impedance measurement and a three-terminal impedance measurement. In some embodiments, if the two-terminal measurement circuit 400′ is active, then the three-terminal measurement circuit 402 is inactive. Likewise, if the three-terminal measurement circuit 402 is active, then the two-terminal measurement circuit 400′ is inactive.

A benefit of this approach is that for application in which two-terminal impedance measurements provide improved specificity and/or sensitivity the two-terminal impedance measurement circuit 400′ may be utilized with respect to the first electrode E₁ and for applications in which three-terminal impedance measurements provide improved specificity and/or sensitivity the three-terminal impedance measurement circuit 402 may be utilized.

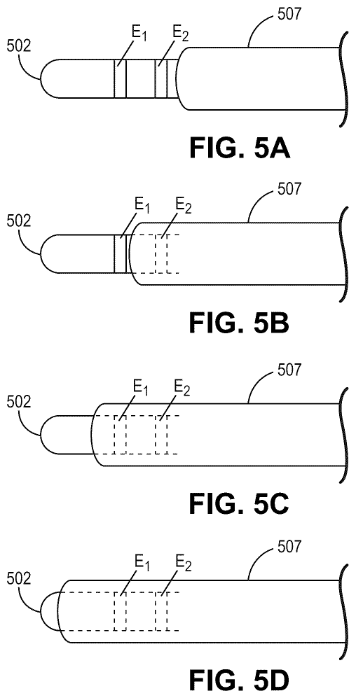

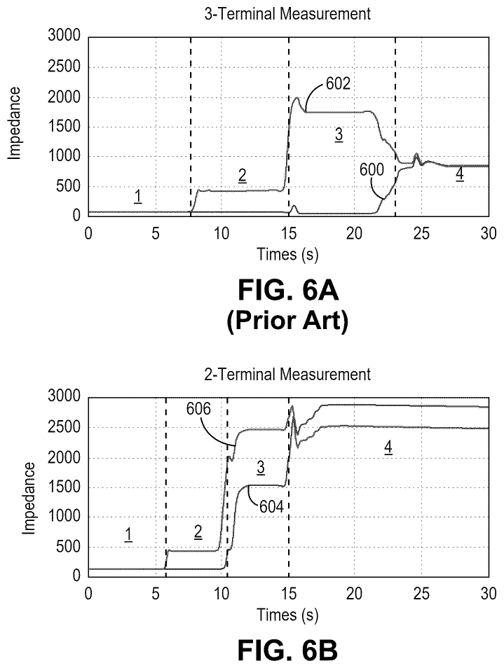

FIGS. 5A-5D illustrate a medical device 502 having a pair of shaft electrodes E₁ and E₂ sheathed within sheath 507 according to some embodiments. FIG. 6A and FIG. 6B are graphs illustrating three-terminal impedance measurements and two-terminal impedance measurements taken during the various stages of sheathing the medical device 502 within the sheath 507.

FIG. 5A illustrates the medical device 502 in a first stage, in which none of the two shaft electrodes E₁ and E₂ are located out of sheath 507. FIG. 5B illustrates the medical device 502 in a second stage, in which medical device 502 has been partially sheathed, with shaft electrode E₂ being located within the sheath 507 (as indicated by the dashed lines) while shaft electrode E₁ remain unsheathed. FIG. 5C illustrates the medical device 502 in a third stage, in which medical device 502 has been further sheathed, with shaft electrodes E1 and E2 being located within the sheath 507 Finally, FIG. 5D illustrates the medical device 502 in a fourth or final stage, in which medical device 502 has been more fully sheathed, wherein both shaft electrodes E₁ and E₂ are located further within the sheath 507.

FIG. 6A illustrates three-terminal impedance measurements taken with respect to the shaft electrodes E₁ and E₂ during the various stages of sheathing the medical device 502 within sheath 507 shown in FIGS. 5A-5D. Measurement 600 is for distal electrode E1 and measurement 602 is for proximal electrode E2. The three-terminal measurement circuit 200 known in the prior art and shown in FIG. 2 may be utilized to make the distal and proximal three-terminal measurements shown in FIG. 6A.

In stage one (FIG. 5A) none of the electrodes is sheathed and both the distal and proximal three-terminal measurements 600, 602, respectively, reflect the low impedance path to the surface reference electrode. In stage 2 (FIG. 5B), the proximal electrode E₂ is sheathed within the sheath 507 and the proximal three-terminal measurement 602 shows an increase in impedance while the distal three-terminal measurement 600 corresponding to unsheathed electrode E₁ remains unchanged. In stage 3 (FIG. 5C), both electrodes E₁ and E₂ are sheathed. The proximal three-terminal measurement 602 increases again from stage 2, while the distal three-terminal measurement 600 remains unchanged (despite electrode E1 being sheathed). In stage 4 (FIG. 5D), both electrodes E₁ and E₂ are sheathed more fully within the sheath. The proximal three-terminal measurement 602 taken decreases from stage 3, while the distal three-terminal measurement 600 increases. This shows that the three terminal measurement does not capture the status of E1 accurately, particularly when it is just sheathed.

FIG. 6B illustrates two-terminal impedance measurements taken with respect to patient reference electrode during the various stages of sheathing the medical device 502 within sheath 507 shown in FIGS. 5A-5D. The proximal measurement 606 is a two-terminal measurement taken of electrode E2 and distal measurement 604 is a two-terminal measurement of electrode E1. The two-terminal measurement circuit 300 shown in FIG. 3 may be utilized to make the distal and proximal two-terminal measurements shown in FIG. 6B.

In stage one (FIG. 5A) none of the electrodes is sheathed and both the distal and proximal two-terminal measurements 604, 606, respectively, are low impedance. In stage 2 (FIG. 5B), the proximal electrode E₂ is sheathed within the sheath 507 and the proximal two-terminal measurement 606 corresponding with proximal electrode E₂ shows an increase in impedance while the distal two-terminal measurement 604 corresponding with unsheathed electrode E₁ remains unchanged. In stage 3 (FIG. 5C), electrodes E₁ and E₂ are sheathed. The proximal two-terminal measurement 606 increases again from stage 2, while the distal two-terminal measurement 604 increases from stage 2. In stage 4 (FIG. 5D), both electrodes E₁ and E₂ are sheathed. The proximal two-terminal measurement 606 taken with respect to sheathed electrode E₂ increases slightly from stage 3, while the distal two-terminal measurement 604 from stage 3.

One of the benefits of the two-terminal impedance measurements during sheathing of the medical device is the more responsive and monotonic changes in impedance as compared with the three-terminal measurements. For example, the proximal two-terminal measurement 606 associated with electrode E₂ increases in stage 2 in response to the proximal electrode E₂ being sheathed. The proximal two-terminal measurement 606 increases again in both stages 3 and 4 as the sheathing process continues and the proximal electrode E₂ is positioned further within the sheath 507. Likewise, the distal two-terminal measurement 604 corresponding with electrode E₁ remains unchanged (low impedance) when stages 1 and 2 when the distal electrode E₁ remains unsheathed, but increases in each of stages 3 and 4 in response to the distal electrode E₁ being sheathed. In some embodiments, this monotonic increase in the two-terminal measurement allows the two-terminal measurement to be utilized not only to determine whether or not an electrode has been sheathed, but the position of the electrode within the sheath, with the two-terminal impedance measurement increasing (at least to a point) as the electrode is positioned further within the sheath. Another benefit associated with utilizing two-terminal measurements over three-terminal measurements is that the number of shaft electrodes required may be reduced as the two-terminal measurement only requires a single shaft electrode (as opposed to two shaft electrodes for three-terminal measurements).

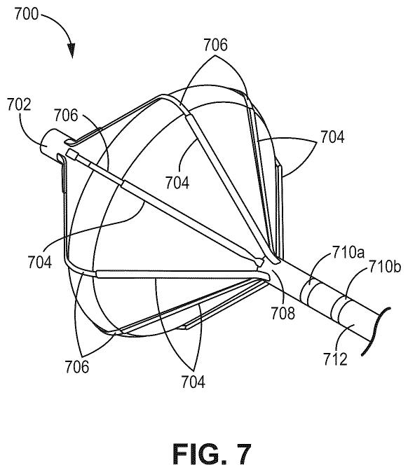

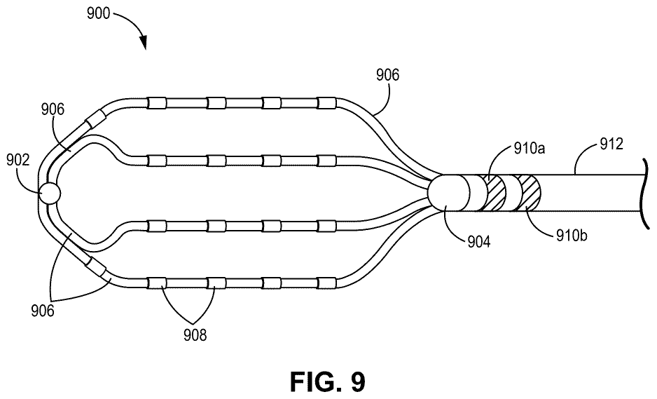

Although FIG. 6B is described with respect to the shaft electrodes E₁ E₂ shown in FIGS. 5A-5D, sheath detection using two-terminal measurements may be utilized on a number of different types of catheters that include shaft electrodes. For example, FIG. 7 illustrates a catheter having a basket-shaped distal end along with at least one shaft electrode 710a, 710b located proximal of the basket along the shaft. Likewise, FIG. 9 illustrates a catheter having a planar array of splines and electrodes along with at least one shaft electrode 910a, 910b located proximal of the planar array of splines along the shaft 912. One or more of these shaft electrodes may be utilized for sheath detection as illustrated with respect to FIG. 6B. One of the benefits of utilizing two-terminal measurements

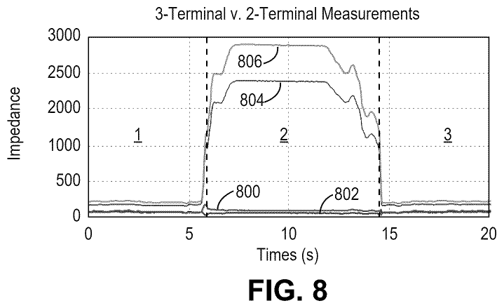

FIG. 7 is an oblique view of a distal end of a catheter 700 having a plurality of splines 704 in a basket configuration according to some embodiments. In the embodiment shown in FIG. 7 each of the plurality of splines 704 is connected between a distal end 702 and a proximal end 708 that in turn is attached to a shaft 712. In some embodiments, each of the plurality of splines includes an electrode 706. In other embodiments, each spline may include more than one electrode. Likewise, in some embodiments one or more shaft electrodes 710 are located along shaft 712. Spline electrodes 706 may be utilized in a variety of ways, including for localization, mapping, and delivery of therapy. In some embodiments, spline electrodes 706 may also be utilized to make two-terminal impedance measurements. As described in other embodiments, the two-terminal impedance measurements may be utilized for sheath detection and/or for tissue proximity/contact. FIG. 8 compares two-terminal measurements of the spline electrodes for sheath detection with three-terminal measurements taken with respect to adjacent spline electrodes.

FIG. 8 is a graph comparing three-terminal measurements with two-terminal measurements associated with spline electrodes 706 located on the plurality of splines 704. Lines 800 and 802 represent three-terminal measurements taken with respect to one of spline electrodes 706 located on respective splines 704. Lines 804 and 806 represent two-terminal measurements taken with respect to first and second splines electrodes 706 located on respective splines 704. The graph is divided into first, second and third stages. During the first stage (denoted with the numeral ‘1’) the basket assembly comprised of the plurality of splines is unsheathed. At the transition between the first stage and the second stage the basket assembly is sheathed, including the spline electrodes 706 located on the splines 704 of the basket assembly. The basket assembly and corresponding spline electrodes 706 remain sheathed during the second time period. At the transition between the second stage and the third stage the basket assembly is unsheathed, including the spline electrodes 706.

As shown in FIG. 8, the three-terminal measurements 800, 802 do not provide a noticeable change between the first stage, the second stage, and the third stage. In contrast, the two-terminal measurements 804 and 806 provide a very noticeable and easily detectable change in measured values between the first stage, the second stage, and the third stage. In embodiments in which each of the splines 704 include a single spline electrode 706, each of the spline electrodes 706 will be sheathed at approximately the same time. Therefore, in some embodiments, sheath detection may only require one of the plurality of spline electrodes 706 to be monitored using a two-terminal measurement. In some embodiments, the other spline electrodes may be utilized to make three-terminal measurements depending on the application. In some embodiments, the electrode utilized to make two-terminal measurements may also be utilized as part of an electrode pair utilized to make three-terminal measurements (as described with respect to FIG. 4B, above).

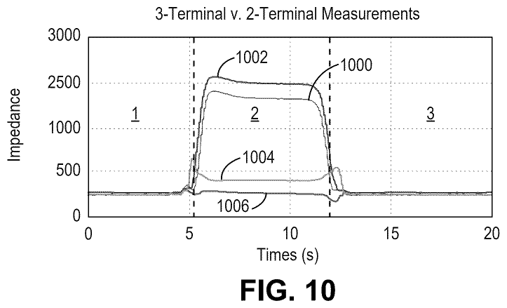

FIG. 9 is a top view of a distal end of a catheter 900 having a plurality of splines 906 located in a plane according to some embodiments. In the embodiment shown in FIG. 9 each of the plurality of splines 906 is connected between a distal end 902 and a proximal end 904 that in turn is attached to a shaft 912. In some embodiments, each of the plurality of splines 906 includes a plurality of spline electrodes 908 arranged in an array. Likewise, in some embodiments one or more shaft electrodes 910 are located along shaft 912. Spline electrodes 908 may be utilized in a variety of ways, including for localization, mapping, and delivery of therapy. In some embodiments, spline electrodes 908 may also be utilized to make two-terminal impedance measurements. As described in other embodiments, the two-terminal impedance measurements may be utilized for sheath detection and/or for tissue proximity/contact. FIG. 10 compares two-terminal measurements of the spline electrodes with three-terminal measurements taken with respect to adjacent spline electrodes.

FIG. 10 is a graph comparing three-terminal measurements with two-terminal measurements associated with spline electrodes 908 located on the plurality of splines 906. Lines 1004 and 1006 represent three-terminal measurements taken with respect to one pair of spline electrodes 908 located on respective splines 906. Lines 1000 and 1002 represent two-terminal measurements taken with respect to first and second splines electrodes 908. The graph is divided into first, second and third stages. During the first stage (denoted with the numeral ‘1’) the planar array assembly comprised of the plurality of splines is unsheathed. At the transition between the first stage and the second stage the planar array assembly is sheathed, including the spline electrodes 908 located on the splines 906 of the assembly. The planar array assembly and corresponding spline electrodes 908 remain sheathed during the second time period. At the transition between the second stage and the third stage the planar array assembly is unsheathed, including the spline electrodes 908.

As shown in FIG. 10, the three-terminal measurements 1004 provides a slightly increased impedance in response to the spline electrodes 908 being sheathed while three-terminal measurement 1006 does not provide a noticeable change in impedance in response to the spline electrode being sheathed. In contrast, the two-terminal measurements 1000 and 1002 provide a very noticeable and easily detectable change in measured values between the first stage, the second stage, and the third stage. In the embodiment shown in FIG. 10, both of the spline electrodes 908 being monitored are located at approximately the same location along the length of the spline, so both electrodes 908 are sheathed at approximately the same time. In other embodiments, two-terminal measurements may be made with respect to electrodes located at different longitudinal positions along the spline 906 and can be utilized to detect the position of the planar array relative to the sheath.

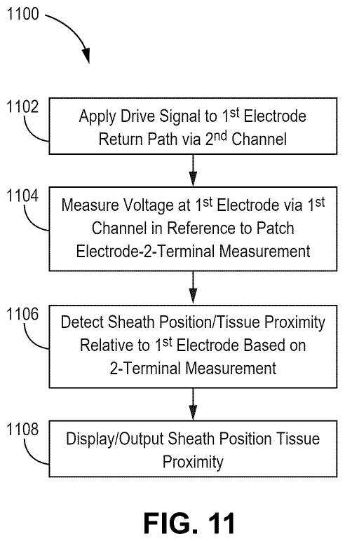

FIG. 11 is a flowchart illustrating a method of detecting sheath position and/or tissue proximity based on one or more two-terminal measurements according to some embodiments. At step 1102 drive signals are applied to a first electrode. In some embodiments, the two-terminal measurement circuit comprises first and second channels and drive circuitry connected to provide a drive signal to the first electrode. A return path for the drive circuitry is provided by the second channel of the two-terminal measurement circuit.

At step 1104, the first channel is utilized to measure a voltage associated with the first electrode. The voltage is measured in response to the drive signal—and in particular the drive current—provided by the drive circuitry. The first channel compares the voltage measured at the first electrode with a reference voltage provided by a reference electrode. In some embodiments the reference electrode is a patch electrode located on the skin of the patient. In other embodiments the reference electrode is located on the medical device that also includes the first electrode. In other embodiments, the reference electrode is located on a second medical device. In some embodiments, the comparison of the voltage at the first electrode and the reference voltage results in a voltage representative of the difference or differential between the respective inputs and is related to an impedance associated with the first electrode. In some embodiments, the voltage generated by the comparison is representative of the impedance between the first electrode and the reference electrode, and can be utilized to detect aspects the surrounding environment. In some embodiments, the monitored voltage generated by the comparison is referred to as the two-terminal measurement. In other embodiments, the monitored voltage may be utilized in combination with information regarding the magnitude of the current provided to the first electrode to calculate an impedance value associated with the first electrode. Either way, the term two-terminal measurement refers to the two voltages monitored at their respective terminals (i.e., node associated with the first electrode and the reference node associated with the reference electrode).

At step 1106, the two-terminal measurement measured at step 1104 is utilized to detect sheath position relative to the first electrode and/or tissue proximity/contact status relative to the first electrode. Detection of these conditions may be based on a comparison of the two-terminal measurement to one or more threshold values utilized to detect a condition. For example, an increase in the measured two-terminal measurement beyond a threshold may be utilized to detect the sheathing of the electrode making the two-terminal measurement.

At step 1108 an output is generated indicating the detected condition. For example, a determination at step 1106 that the electrode has been sheathed is displayed to the user. The display may include color-coding of the electrode to indicate sheathing status and/or tissue proximity/contact or may include other methods of displaying attributes of the electrodes. In some embodiments, a two-terminal measurement made with respect to a first electrode may be utilized to make decisions regarding the sheathing status and/or tissue proximity/contact of other electrodes. For example, a determination that a first spline electrode located on a basket electrode has been unsheathed provides information that the plurality of other spline electrodes have also been unsheathed.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A medical device system comprising:
a reference electrode;
drive circuitry configured to provide drive signal;
a medical device having a first electrode located on the medical device, the first electrode being connected to receive the drive signal from the drive circuitry; and
sense circuitry having a first channel and a second channel, the first channel comparing a first voltage measured at the first electrode and a reference voltage measured at the reference electrode and the second channel providing a return path for the drive circuitry.

2. The medical device system of claim 1, wherein the drive signal supplied to the first electrode returns to the drive circuitry through the reference electrode and the second channel.

3. The medical device system of claim 1, wherein the second channel is connected to the first channel via a shunt resistor.

4. The medical device system of claim 1, wherein the first channel includes an amplifier having a first input connected to monitor voltage on the first electrode and a second input connected to the reference electrode.

5. The medical device system of claim 4, wherein an output of the amplifier is representative of impedance between the first electrode and the reference electrode.

6. The medical device system of claim 1, wherein the second channel includes a second amplifier having a first input connected to the reference electrode through a ground isolation terminal and a second input connected to the reference electrode.

7. The medical device of claim 1, wherein a circuit path exists from the drive circuitry, to the first electrode, to the reference electrode, to a return on the drive circuitry via a designed return path between the first channel and the second channel.

8. The medical device system of claim 1, wherein the first electrode is located on a shaft of the medical device, on a spline of a basket assembly, or as part of an array of electrodes.

9. The medical device system of claim 1, further including a second drive circuit providing a second drive signal, a third channel and a fourth channel, wherein the third channel is connected to the first electrode and the fourth channel is connected to a second electrode located on the medical device, wherein the second drive signal is provided to both the first electrode and the second electrode and wherein the third channel and the fourth channel make a three-terminal impedance measurement.

10. The medical device system of claim 1, wherein the reference electrode is a surface patch electrode.

11. The medical device system of claim 1, wherein the reference electrode is a second electrode located on the medical device.

12. A two-terminal impedance measurement circuit comprising:
drive circuitry configured to provide a drive signal; and
sense circuitry having a first channel and a second channel, the first channel measuring impedance between a first electrode located on a medical device and a reference electrode and the second channel providing a designed return path for the drive circuitry without the second channel being connected to any electrodes on the medical device.

13. The two-terminal impedance measurement circuit of claim 12, wherein the drive signal provided to the first electrode is provided at a unique frequency.

14. The two-terminal impedance measurement circuit of claim 12, wherein the second channel is connected to the first channel via a shunt resistor.

15. The two-terminal impedance measurement circuit of claim 12, wherein the first channel includes an amplifier having a first terminal connected to monitor voltage on the first electrode and a second terminal connected to a reference voltage associated with the reference electrode.

16. The two-terminal impedance measurement circuit of claim 15, wherein the second channel provides a return path for the drive circuitry via the reference electrode and ground reference shared by the reference electrode and the second channel.

17. The two-terminal impedance measurement circuit of claim 12, wherein the reference electrode is a surface patch electrode or a second electrode located on the medical device.

18. A method of making a two-terminal measurement using a sense circuitry configured for three-terminal impedance measurements, the method comprising:
providing, using drive circuitry, a first drive signal to a first electrode located on a medical device;
measuring, using a first channel a voltage between the first electrode and a reference electrode; and
providing, using a second channel, a return path for the drive circuitry, wherein the second channel is not connected to any electrodes located on the medical device.

19. The method of claim 18, wherein the second channel is connected to the first channel via a shunt resistor.

20. The method of claim 18, wherein the reference electrode is a surface patch electrode or a second electrode located on the medical device.
